(19) United States
(12) Patent Application Publication
(10) Pub. No.: US 2007/0110737 A1
(43) Pub. Date: May 17, 2007

(54) Compositions and method for decreasing the appearance of skin wrinkles

(76) Inventor: Allan Mishra, Menlo Park, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/581,583

(22) PCT Filed: Dec. 23, 2004
(86) PCT No.: PCT/US04/43383

§ 371 Date: Jun. 2, 2006

Related U.S. Application Data
(60) Provisional application No. 60/533,467, filed on Dec. 29, 2003.
(60) Provisional application No. 60/533,328, filed on Dec. 29, 2003.
(60) Provisional application No. 60/533,161, filed on Dec. 29, 2003.

(51) Int. Cl. A61K 35/14

(52) U.S. Cl. 424/93.72

(57) ABSTRACT

Compositions which include platelet-rich plasma and fibroblast cells are disclosed for the treatment of skin. In particular, administration of platelet-rich plasma in a dermatologically acceptable carrier repeatedly to skin is disclosed to reduce the appearance of wrinkles.

### FIELD OF THE INVENTION

The invention relates generally to the field of dermatology and to compositions which may be applied to the skin to obtain beneficial results including a decrease in the appearance of wrinkles.

### BACKGROUND OF THE INVENTION

Physical appearance, beauty, and the desire to maintain youthfulness are concepts that are not new. In all societies around the world, there are easily recognizable incentives regarding the maintenance of a favorable appearance. The nature and form of desirable appearance varies, but each culture has developed its own standards and norms. In our modern society, psychologists, sociologists, and economists, as well as those on Madison Avenue, have made observations about our attitudes regarding appearance. Cosmetologists, health experts, personal trainers, and cosmetic surgeons have supplied us with various means by which we can maintain our youthful appearance and improve the undesired and undesirable changes of aging. Aging and aging of the face are the results of many factors. Some of these are intrinsic, some extrinsic. Some are controllable, some uncontrollable. The rate at which different people age is variable. Aging on a biologic basis is not a homogenous, even-flowing process, but appears to evolve with various accelerations and decelerations. Individuals appear to age at different rates; however, we all age in a similar progression, and therefore discernable patterns emerge.

The aging process is believed to be based on the same principles in every individual. The intrinsic aspects of aging are largely based on heredity; these are programmed into the individual at the cellular level and are largely unalterable. The extrinsic factors are the results of an individual's habits, nutrition, and exposure to deleterious factors, such as cigarette smoking and ultraviolet sunlight. The individual can influence or control the extrinsic factors largely by avoidance and through the maintenance of good health habits and exercise. Once the observable changes of aging have occurred, few are reversible. Many of the changes, however, can be improved through makeup, cosmetic skin care, and cosmetic rejuvenative surgery.

Histologically, sun-damaged epidermis is significantly thickened and disorganized as compared to non-damaged skin. In response to long-term exposure to sun, epidermal melanocytes enlarge, proliferate and migrate to higher levels of the epidermis. Chronic stimulation of melanocyte often leads to dyschromia, spotty hyper pigmentation, and the proliferation of pigmented keratosis. It is also known that ultraviolet radiation causes extensive damage to both cellular and structural components of the dermis.

Nutritionists have long warned about the deleterious effects of free radicals. Indeed it has been well documented that significant damage to biological tissues results from free radical induced oxidation. The presence of oxidation inducing free radicals is promoted by exposure to several environmental factors. Among these factors are air pollutants, ultraviolet radiation, diet and cosmetic agents. Dermatologically, the presence of free radicals promotes and sometimes accelerates the aging process. One result of this acceleration would be the observance of wrinkles. Indeed, similar concerns regarding free radical induced damage to the skin has as well been documented.

The genetically determined process of the aging skin results in a predictive group of morphologic and physiologic changes. In the skin of an aged person, the epidermis is of variable thickness, there is modest diversity in cell size and shape, the dermatoepithelial abutment is flattened and rete ridges are lost, cumulatively rendering aged skin fragile and susceptible to injury from sheering forces. The dermis of senescent skin is characterized by marked cellular atrophy and a corresponding reduction in metabolic activity. As a result, the percentage of newly synthesized collagen in the dermis decreases. As a result, aged or aging skin is less distensible, poorly resilient, and prone to fine wrinkling. Furthermore, in aging skin the epidermis thins with a gradual loss of rete ridges and concomitant decrease in cell turnover in the basal cells. Furthermore, the surface corneocyte layer thickens with age. The dermis also becomes thinner with decreased collagen content, degeneration of elastic fibrils, decreased water content and the gradual addition of stable cross-links in and between collagen fibrils. Skin thickness in women reaches maximum at approximately age 35 and decreases gradually thereafter. In men, the slain thickness versus age curve is different, with the peak in skin thickness occurring at age 45. With these changes, there is a loss of the biomechanical properties of the skin and with advancing age, the ability of the skin to recover from the initial stages of deformation drops. The appearance of aged, sun-damaged skin is therefore the result of these intrinsically and extrinsically caused changes. The skin may have uneven pigmentation and an uneven texture, may be wrinkled, less distensible, and more prone to laxity.

Fine wrinkles generally begin to appear in individuals in their twenties; these wrinkles deepen as individuals approach their thirties. In the upper face, crow's feet and wrinkling above the eyelids may occur as early as the late twenties. The formation of crow's feet is secondary to the contraction of the orbital portion of the orbicularis oculi muscle and they are accentuated by the elevation of the upper cheek by the zygomaticus and the zygomatic head of the quadratus labii superioris muscle.

Chronic exposure to ultraviolet light damages structural and functional components of the skin. The resulting photo-damage, or photo-aging as it is sometimes called, is characterized by wrinkling, sallowness, modeled hyper pigmentation, and laxity. Histologically, photo-damage is accompanied by epidermal thinning, variable atypia, large, irregular grouped melanocyte and elastosis. In 1986, Kligman et al. reported that tretinoan cream (Retinin-A), which had been used for more than twenty years in the treatment of acne vulgaris, could also produce a more attractive, less-wrinkled skin in older patients. When applied to persons with photo-damaged skin, tretinoan cream proved effective in partially reversing structural skin damage. In the past ten years, clinical and histologic studies have confirmed the efficacy of tretinoan as therapy for smoothing skin texture, reducing wrinkles, and improving skin discoloration. Further research by Kligman et al. studied the efficacy of topical tretinoan cream on reversing facial skin photo-damage. Elderly patients received a daily facial application of 0.05% tretinoan cream for six to twelve months and six age-matched subjects received a placebo vehicle only. Although clinical changes were deemed to be slight, many histologic effects were observed in skin biopsy specimens. One of the clinical changes observed from this research included normalization of various skin structures such as thickening of a previously atrophic epidermis, elimination of dysplasia and atypia, and more uniform dispersion of melanin and the formation of new dermal collagen and blood vessels. Furthermore, topical tretinoan has been shown to have beneficial effects in the treatment of hyper pigmented lesions of a variety of types such as those associated with photo-damage in white patients, and those caused by inflammation or melasma in black patients. Additionally, “liver spots” on the face or upper extremities of patients with photo-damage were treated with 0.1% tretinoan cream daily, resulting in a lightening of the liver spots (more appropriately called hyper pigmented macules or also termed actinic lentigines). However, tretinoan frequently induces mild to moderate dermatitis. although, percutaneously absorbed tretinoan has no detectible effect on plasma concentrations of the drug and its metabolites in any of the protocols reported, many patients see the induced mild to moderate dermatitis as prohibitively discomforting for effective use in correction of wrinkles.

Much work has been directed towards the use of topically applied organic acids, which cause a destruction and subsequent removal of the outer dermis layers, thereby provoking the formation of new collagen. It is believed that the induced formation of new collagen would occur preferentially over old collagen, thereby replacing wrinkles with new, young dermis in the absence of said wrinkles. Previous works with chemical peels, as they are known, discuss the post-peel development of a zone of collagen. The zone of collagen is a deposit of a new collagen that is laid down in the upper dermal layers after a chemical peel. Both phenol and trichloracetic acids (TCAs) have been histologically studied to compare the amount of new deposition in the zone of collagen. The deeper depth of necrosis caused by the chemical peeling agent resulted in a deeper zone of collagen. Thus, more damaging chemical peels can smooth deeper layers of the skin. For instance, higher concentrations of tichioracetic acid, perhaps 50-70%, can penetrate to layers of the reticular dermis and also cause a zone of new collagen to that same depth. However, higher concentrations of trichloracetic acid can lead to scarring, and other severe risks involved with trichloracetic acid use. Although it is true that trichloracetic acids may be applied at a lesser concentration, the same types of risks are involved as are present with a higher concentration because of the inherent strength of the acid involved. α-Hydroxy acids (AHAs) have been used for many years as exfoliants, moisturizers, and emollients. Lactic acid salts, most notably sodium lactate, have been hypothesized to be part of the skin's own natural moisturizing system. In addition, AHAs and salicylic acid, a structurally similar β-hydroxy acid, have been used for at least 40 years as peeling agents.

Studies have shown that several AHAs (as well as β-hydroxy and carboxylic acids) in low concentration (5%) stimulate epidermal turnover or cell renewal (exfoliation) and have the potential to irritate the skin. This activity is closely lined to acidic pH as neutralized acids lose their ability to exfoliate the skin.

The moisturizing activity of AHAs and their ability to exfoliate the skin and interfere with intercellular cohesion in the outer epidermis are well documented. It is suggested that AHAs interfere with cohesion in the stratum granulo sum, unlike salicylic acid and other exfoliants.

Several studies on the activity of a buffered 12% ammonium lactate lotion have documented its moisturizing activity (Wehr et al. Controlled two-center study of lactate 12 percent lotion and a petrolatum-based crearm in patients with xerosis, Cutis, 37:205-9 (1986). The effect of moisturizers on skin surface hydration has been measured in vivo by electrical conductivity, Curr. Ther. Res. Clin. Exp., 50:712-9 (1991). The efficacy of 12 percent ammonium lactate has been studied with regard to the treatment of dry skin of the feet. A clinical product review is provided in J. Curr. Podiatr. Med., 37:15-7(1988)). Lavker et al., Effects of topical ammonium lactate on cutaneous atrophy from a potent tropical corticosteroid, J. Am. Acad. Dermatol., 26:535-44 (1992), found that ammonium lactate caused an increase in dermal ground substance and increased glycosaminoglycan synthesis. Murad et. al., The use of glycolic acid as a peeling agent, Dermatological Clinic on Cosmetic Dermatology, Murad H, editor, Philadelphia: W B Saunders, (1995), demonstrated that aggressive glycolic acid peels significantly increase collagen and dermal ground substance. Precisely how and why AHAs produce these effects is not known.

Vitamin C (ascorbic acid) is alleged to protect the brain and spinal cord from free radicals. It promotes collagen (connective tissue) synthesis, lipid (fat) and carbohydrate metabolism, and the synthesis of neurotransmitters. It is also essential for optimum maintenance of the immune system. Vitamin C is toxic to a wide range of cancer cells, especially melanoma. The oxidizing enzyme tyrosine that catalyzes the aerobic action of tyrosine into melanin and other pigments is also inhibited by the presence of Vitamin C. Vitamin C has been found to be effective in catalyzing the immune response to many viral and bacterial infections. Besides the many applicable uses set forth above, Vitamin C is essential for collagen synthesis and wound healing. International patent application, WO 96/14822, published May 23, 1996, and corresponding U.S. Pat. No. 5,785,978, issued Jul. 28, 1998, teach that concentrated dry powdered antioxidants, Vitamin C and its salts in particular, may be compounded with adhesives and applied to target areas where wrinkles develop to ameliorate photo, oxidative and stress damage and improve skin appearance. However, it is to be noted that only the use of ascorbic acid is shown and the adhesive/ascorbic acid compositions illustrated resulted in adhesive remaining on the skin of the wearer when a patch containing the adhesive composition was removed. Also, application of such patches to sensitive areas, such as around the eyes, often results in pain and trauma during the removal process.

Modern environmental conditions, such as heating and air conditioning, exposure to the sun, and environmental pollution exert severe stress on the skin and accelerate the natural aging process resulting in wrinkles, decreased firmness and elasticity, dryness and other cosmetically undesirable effects. Although a number of skin cream compositions already exist, there is a need for a simple-to-apply and effective all-in-one cosmetic treatment, such as a skin preparation that can counteract and minimize, simultaneously, distresses on the skin and improve firmness and elasticity while it counteracts dryness so that wrinkles and other undesirable effects appearing on the skin are corrected or at least delayed.

The present invention utilizes the combination of components present in platelet-rich-plasma (PRP) to obtain beneficial effect in rejuvenation skin. While whole blood may contain about 95% red blood cells, about 5% platelets and less than 1% white blood cells, PRP may contain 95% platelets with 4% red blood cells and 1% white blood cells. PRP can be combined with activating agents such as thrombin or calcium which activate the platelets to release their contents such as cytokinins and other growth factors. PRP has been used in medicine, primarily in bone grafting and dental implant applications and as part of a composition to use as a surgical adhesive. For example, Landesberg et al (U.S. Pat. No. 6,322,785) disclose and autologous platelet gel that includes a PRP for bone grafts and dental implants.

Antanavich et al. (U.S. Pat. No. 5,585,007) disclose preparation of PRP and use as a tissue sealant. Cochrum (U.S. Pat. No. 5,614,214) discloses a biopolymer that optionally includes PRP and its use to temporarily block arteries and veins. Gordinier et al. (U.S. Pat. No. 5,599,558) disclose a platelet releasate product, which includes platelets buffered to approximately pH 6.5, for use in a topical application to wounds.

In view of this background and the surrounding business and medical environment the following invention is presented.

### SUMMARY OF THE INVENTION

A method of treating human skin utilizing platelet-rich-plasma (PRP) is disclosed.

Platelets are concentrated (e.g. out of human blood) and formulated into a pH balanced, dermatologically acceptable composition which may comprise a skin permeation enhancer. The formulated composition may be regularly and repeatedly applied to skin over a period of time until desired results are obtained. The PRP can be used to enhance the growth of human cells such as fibroblasts which fibroblasts, other cells, and/or collagen can be formulated separately or with PRP and applied to human skin and/or injected just below the skin.

An aspect of the invention is a dermatologically acceptable formulation of PRP which formulation may be topical or injectable.

Another aspect of the invention is a method of improving the appearance of wrinkled, lined, dry, flaky, aged or photodamaged skin and improving skin thickness, elastiticity, flexibility by administering, e.g. repeatedly applying an effective amount of a dermatologically acceptable, topical composition of the invention to human skin.

In another aspect of the invention blood is drawn from a patient and PRP obtained from the blood, which PRP is formulated into a pH balanced formulation which is applied to and/or injected under the skin of the same patient from which the blood is drawn.

In yet another aspect of the invention fibroblast cells obtained from a patient are grown on a medium comprising PRP and the resulting fibroblasts are formulated and administered to the patient (e.g. the same patient) topically or by injection into and just below the skin.

Another aspect of the invention is to provide cosmetics comprising a PRP and/or fibroblast cell formulation of the invention.

Still another aspect of the invention is to formulate the PRP and/or fibroblast cell compositions of the invention with other compounds and compositions useful in the treatment of skin, e.g., collagen with PRP and/or fibroblast compositions of invention or PRP formulation with vitamin A (retinol) and/or, vitamin E in a pH balanced dermatologically acceptable carrier.

Another aspect of the invention is to formulate PRP and/or PRP releasate with a skin permeation enhancer for single or repeated applications to the skin of the same patient from which the PRP was obtained from to obtain any desired results including promoting the growth of endogenous cells just below the outer layer of skin and/or reducing wrinkles and/or reducing the appearance of wrinkles.

Yet another aspect of the invention is a formulation of a platelet releasate fractionated to remove or isolate at least a portion of certain components and to combine the fractionated releasate with one or more skin permeation enhancers for application to human skin.

A method of doing business is disclosed whereby a patient has blood extracted and the patient's blood is used to create a platelet rich plasma (PRR) formulation which is pH balanced in a dermatologically acceptable formulation. The formulation may be sold to the patient for topical application by the patient. The business which extracts the blood and formulates the composition comprising the PRP may counsel the patient other aspects of skin care including diet, exercise, other skin care products and other methods used to improve health and appearance of skin.

The method of doing business may further provide the patient with counseling and/or actual procedures and products as regards to sun screens, LASER treatments, anti-oxidants, including α-lipoic acid, vitamins A, C and E, collagen injection, Botox®, chemical peels, with compounds such as phenol trichloacetic acids (TACs), skin abrasions, α-hydroxy acids, smoking cessation programs and the like.

These and other aspects of the invention will become apparent to those skilled in the art upon reading this disclosure.

### BRIEF DESCRIPTION OF THE DRAWINGS

The invention is best understood from the following detailed description when read in conjunction with the accompanying drawing. It is emphasized that, according to common practice, the various features of the drawings are not to-scale. On the contrary, the dimensions of the various features are arbitrarily expanded or reduced for clarity. Included are the following figures:

FIG. 1 is a graph of cell count versus time for cultured fibroblast cells in PRP.

FIG. 2 is a graph of cell count for three different concentrations of PRP releasate and a control.

FIG. 3 is a graph of cell counts over seven days for a control and a culture with sonocated PRP.

FIG. 4 is a first type of transdermal patch, according to the invention, with a permeable membrane.

FIG. 5 is a second type of transdermal patch, according to the invention, without a permeable membrane.

### DETAILED DESCRIPTION OF THE PREFERRED EMBODIMENT

Before the present compositions, formulations and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure controls to the extent it contradicts an incorporated publication.

It must be noted that as used herein and in the appended claims, the singular forms “a”, “an”, and “the” include plural referents unless the context clearly dictates otherwise. Thus, for example, reference to “a platelet” includes a plurality of such platelets and reference to “the carrier” includes reference to one or more carriers and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “platelet” is used here to refer to a blood platelet. A platelet can be described as a minisule protoplasmic disk occurring in vertebrate blood. Platelets play a role in blood clotting. The platelet may be derived from any source including a human blood supply, or the patient's own blood. Thus, the platelets in the composition of the inventions may be autologous. The platelets may be homologous, i.e. form a human but not the same human being treated with the composition.

The terms “treatment”, “treating” and the like are used herein to generally mean obtaining a desired pharmacologic, physiologic or cosmetic effect. The effect may be prophylactic in terms of completely or partially preventing a condition, appearance or symptom thereof and/or may be therapeutic in terms of a partial or complete cure for a condition and/or adverse effect attributable to a condition or disease. “Treatment” as used herein covers any treatment of a condition, disease or undesirable appearance in a mammal, particularly a human, and includes:
(a) preventing the disease, condition or appearance such as wrinkles from occurring in a subject which may be predisposed to such but has not yet been observed or diagnosed as having it;
(b) inhibiting the disease, condition or appearance, i.e., causing regression of condition or appearance.
(c) relieving the disease, condition or undesired appearance, i.e., causing regression of condition or appearance.

The invention is directed toward treating patients with skin diseases, undesirable skin conditions or appearances and is particularly directed toward treating older skin to provide a younger appearance, i.e., preventing, inhibiting or relieving the effects of aging on skin and thereby improving the appearance of wrinkled, lined, dry, flaky, aged or photodamaged skin and improving skin thickness, elasticity, flexibility and/or plumpness at one or more particular sites. More specifically, “treatment' is intended to mean providing a therapeutically detectable and beneficial effect on a patient suffering from a skin condition or appearance which the patient has found to be undesirable.

The terms “synergistic”, “synergistic effect” and like are used herein to describe improved treatment effects obtained by combining one or more active components together in a composition or in a method of treatment. Although a synergistic effect in some field is meant an effect which is more than additive (e.g., 1+1=3) in the field of skin treatment an additive (1+1=2) or less than additive (1+1=1.6) effect may be synergistic. For example, if one active ingredient removed 50% of a wrinkle and a second active ingredient removed 50% of a wrinkle the combined (and merely additional) effect would be 100% removal of the wrinkle. However, the effect of both would not be expected to remove 100% of the wrinkle. Often, two active ingredients have no better or even worse results than either component by itself. If an additive effect could be obtained with such treatments than multiple ingredients could be applied to completely remove all wrinkles and such is not the case.

The term “platelet-rich-plasma,” “PRP” and the like are used interchangeable here to mean a concentration of platelets in a carrier which concentration is above that of platelets normally found in blood. For example, the platelet concentration may be 5 times, 10 times, 100 times or more the normal concentration in blood. The PRP may use the patient's own plasma as the carrier and the platelets may be present in the plasma at a range of from about 200,000 or less to 2,000,000 or more platelets per cubic centimeter. The PRP may be formed from whole blood e.g. by technology disclosed in any of 5,614,106; 5,580,465; 5,258,126 or publication cited in these patents and if needed stored by technology as taught in 2002/0034722A1; 5,622,867 or publications cited therein. The PRP may comprise blood component other than platelets. It may be 50% or more, 75% or more, 80% or more, 95% or more, 99% or more platelets. The non-platelet components may be plasma, white blood cells and/or any blood component. PRP is formed from the concentration of platelets from whole blood, and may be obtained using autologous, allogenic, or pooled sources of platelets and/or plasma. PRP may be formed from a variety of animal sources, including human sources.

The “dose” of platelets administered to a patient will vary over a wide range based on the age, weight, sex and condition of the patient as well as the patients' own normal platelet concentration, which as indicated above can vary over a ten fold or greater range. Doses of 1 million to 5 million platelets are typical but may be less or greater than such by a factor of two, five, ten or more.

The term “platelet releasate” is the PRP as defined above but treated so that what is inside the platelet shells is allowed to come out. The releasate may be subjected to processing whereby the platelet shells are removed and/or other blood components are removed, e.g. white blood cells and/or red blood cells or remaining plasma is removed. The pH of the platelet releasate may be adjusted to physiological pH or higher or to about 7.4±10%, 7.4±5%, 7.4±2% or 7.4 to 7.6 as needed.

“Fractionated platelet releasate” is a portion of a platelet releasate, e.g. a single protein removed from the platelet releasate and a portion includes the remainder which has had the single protein removed. It is understood that when a component is removed it may not be completely removed and that a removed protein may not be completely pure. The technology available at the time will determine the level of removal and/or purity.

The term “iontophoresis” means the migration of ionizable molecules through a medium driven by an applied low level electrical potential. This electrically mediated movement of molecules into tissues and in particular into the skin is in addition to the movement obtained via concentration gradient dependent diffusion. If the tissue (e.g. skin) through which the molecules travel also carries a charge, some electro-osmotic flow occurs. However, generally, the rate of migration of molecules with a net negative charge towards the positive electrode and vice versa is determined by the net charge on the moving molecules and the applied electrical potential. The driving force may also be considered as electrostatic repulsion. Iontophoresis usually requires relatively low constant DC current in the range of from about 2-5 mA. For enhancing the delivery of a formulation of the invention such and a platelet releasate through the skin (transdermal iontophoresis), one electrode is positioned over the treatment area and the second electrode is located at a remote site, usually somewhere else on the skin. The return electrode may, for certain applications, be placed elsewhere on the skin as the iontophoretic delivery electrode. With the present invention the return electrode may be similarly positioned on the skin. The applied potential for iontophoresis will depend upon number of factors, such as the electrode configuration and position on the tissue (skin), the nature and charge characteristics of the molecules (e.g. releasate formulation) to be delivered, and the presence of other ionic species within components of the patch and in the tissue extracellular compartments.

As used herein “Collagen” means pharmaceutical grade collagen used in the treatment of human patients. Collagen is a fibrous protein that form fibrils having a very high tensile strength and that has been found in most multicellular organisms. Collagen serves to hold cells and tissues together and to direct the development of mature tissue. Collagen is the major fibrous protein in skin, cartilage, bone, tendon, blood vessels and teeth.

There are many types of collagen which differ from each other to meet the requirements of various tissues. Some examples of types of collagen are as follows: type one [α1(I)]₂α2 which is found in skin, tendon, bone and cornea; type two [α1(II)₃ which is found in cartilage intervertebral disc, and the vitreous body; type three [α1 (III)]₃ which can be found in skin and the cardiovascular system; type four [α1(IV)]₂α2(IV) which can be found in basement membrane; type five [α1(V)]₂α2(V) and α1(V)α2(V)α3(V) which is found in the placenta and cornea. Examples of newly identified forms of collagen include: type seven (VII) which is found in anchoring fibrils beneath many epithelial; and types nine (IX), ten (X) and eleven (XI), which are minor constituents of cartilage.

The chemical characterization of native collagen was difficult since its low solubility made isolation of collagen a tedious task. Eventually, it was discovered that collagen from tissues of young animals was not as extensively cross linked as that of mature tissues and thus was more amenable to extraction. For example, the basic structural unit of type I collagen, tropo-collagen, can be extracted in intact form from some young, collagen-containing animal tissues.

Substantial information can be found in patents and publications relating to uses of Collagen. For example, see U.S. Pat. Nos. 4,294,241; 4,668,516; 5,640,941; and 5,716,411 all of which are incorporated herein by reference as are the publications and patents cited in these patents to disclose and describe various ways of using collagen which can in turn be mixed with and administered and used with platelet formulations of the present invention.

The terms “permeation enhancer”, “skin permeation enhancer” and the like are used interchangeably here to mean any compound or group of compounds which increase the rate at which a component moves through the skin. The enhancer may have physical and/or chemical characteristics which enhance permeation through the skin. The enhancer may be a natural compound (e.g. a plant polar lipid as taught in U.S. Pat. No. 6,346,244) or a synthetic compound (e.g. a ceramic hydroxyapatite with particles of about 2 to about 6 micrometers in mean diameter as taught in U.S. Pat. No. 6,096,324) and may be used in combination with other materials such as lipid vesicles as taught in U.S. Pat. No. 4,761,288 —see also U.S. Pat. Nos. 5,059,426; 6,238,933; and 5,762,956 which teach permeation enhancers of various types.

The skin permeation enhancers utilized in the present invention may comprise any of or a combination of any of dimethyl sulfoxide (DMSO), a fatty alcohol ester of lactic acid and lower (lower maens 1 to 4 cabons) alkyl ester of lactic acid. Preferably, the enhancer is a mixture of DMSO with lauryl lactate (available as Ceraphil 31 from Van Dyk Chem. Co., Belleville, N.J.) and ethyl lactate. Formulations of the invention may comprise one or a combination of skin permeation enhancers homogeneously dispersed in a formulation of when used on a patch be present in an adhesive polymer matrix. The skin permeation mixture may be present in the adhesive polymer matrix in an effective amount of up to about 30-60% w/w of the total matrix, i.e., at about 35-55% w/w of the matrix.

A skin permeation enhancer may be chosen from any of sodium lauryl sulfate, dibutyl adipate, isopropylmyristate, dimethylsulfoxide, decylmethylsulfoxide, dimethylformamide, dimethylacetamide, glycerylmonocaprylate, propylene glycol, N-alkyl-2-pyrrolidone, d-limonene, menthone, ethanol, and mixtures or combinations thereof.

A skin permeation accelerator may be any common one without particular limitation and it may or may not exert any other influence. Some skin permeation accelerators include, for example, alcohols and polyhydric alcohols such as ethanol, propylene glycol, 1,3-butanediol, and 1,2,6-hexanetriol; fatty acids such as lactic acid, oleic acid, linolic acid, and myristic acid, and their esters; and animal oil, vegetable oil, and a terpene compound such as peppermint oil, 1-menthol, d1-camphor, and N-methyl-2-pyrrolidone.

### Invention in General

Formulations of the invention can be applied topically to and/or injected into and/or under the skin. The formulations comprise platelet and/or fibroblast cells. The platelets and fibroblast cells are preferably obtained from the patient to which the formulation is being administered. A formulation of the invention can be administered to any skin, e.g. to wrinkled, lined, dry, flaky, aged, and photodamaged skin. A range of beneficial results may be obtained, e.g. improving skin thickness, decreasing wrinkles and/or the appearance of wrinkles, improving the elasticity, flexibility and overall appearance.

A formulation of the invention may be produced by drawing blood from a human; and centrifuging the blood to obtain a plasma-rich fraction or PRP. The platelet-rich plasma is then combined with a dermatologically acceptable carrier.

In an aspect, the invention relates to the method wherein the platelet composition is at or above physiological pH. In an aspect, the invention relates to the method wherein the platelet composition optionally includes platelet releasate. In an aspect, the invention relates to the method further comprising: mixing into the platelet composition one or more of the ingredients selected from thrombin, epinephrine, collagen, calcium salts, pH or adjusting agents. Also useful are materials to promote degranulation or preserve platelets, additional growth factors or growth factor inhibitors, small molecule pharmaceuticals such as NSAIDS, steroids, and anti-infective agents.

In an aspect, the invention relates to the method with the proviso that the platelet composition is substantially free from exogenous activators prior to its administration onto or into the skin.

### Additional Active Components

There are a number of compounds which can have a beneficial effect on treating skin. The effect of those components can be enhanced when combined in a composition of the invention. The formulation of the invention may be mixed with and/or administered separately with collagen in any treatment (e.g. wound healing, wrinkle removal and lip enhancement) and one or more other active compounds may be added. For example, further beneficial results may be obtained by combining the compositions according to the invention with at least one substance chosen from vitamins, particularly the vitamins of group A (retinol) and group C and derivatives thereof such as the esters, especially the palmitates and propionates, tocopherols, xanthines, particularly caffeine or theophylline, retinoids, particularly vitamin A acid, extracts of Centella asiatica, Asiatic and madecassic acids and glycosylated derivatives thereof such as asiasticoside or madecassoside, extracts of Siegesbeckia orientalis, extracts of Commiphora mukl and extracts of Eriobotrya japonica, cosmetically acceptable silicon derivatives such as polysiloxanes, silanols and silicones, C₃ -C₁₂ aliphatic alpha-keto acids, particularly pyluvic acid, C₂ -C₁₂ aliphatic alpha-hydroxy acids, particularly citric acid, glycolic acid, malic acid and lactic acid, lipoic acid, amino acids, particularly arginine, citrulline and threonine, ceramides, glycoceramides, sphinogosine derivative, particularly type II and III ceramides, phospholipids, forskolin and derivatives thereof, extracts of Coleus, extracts of Tephrosia, elastase inhibitors, particularly ellagic acid and soya peptides, collagenase inhibitors, particularly plant peptides and extracts such as extracts of roots of Coptidis and extracts of roots of Scutellaria baicalensis Georgi, flavonoids such as wogonin, baicalin and abaicalein, aqueous-ethanolic extracts of leaves of Ginkgo biloba, Mosla chinensis, Salvia officinalis and Cinnamommum cassia, catechuic extracts of Camellia sinensis and aqueous extracts of bean shells of Theobroma cacao, anti-inflammatories, particularly phospholipase A2 inhibitors, soothing agents, particularly extracts of liquorice, glycyrrhetinic acid and ammonium glycyrrhizinate, hydrating agents, particularly polypols, propylene glycol, butylenes glycol, glycerol and hyaluronic acid, agents for combating stretch marks, particularly extracts of horse chestnut and escin, agents for protecting or improving the microcirculation, particularly bioflavonoids from Ginkgo biloba, isodon, extracts if Ami visnaga, visnadine and ruscogenin, free radical inhibitors, particularly polyphenols such as PCO (procyanidolic oligomers) and derivatives thereof and plant extracts, particularly extracts of Curuma longa, antiseborrhea agents, such as a 5-alpha-reductase inhibitor, particularly an extract of Pygeum africanum, and stimulants of the microcirculation of the blood, such as cepharanthine and methyl nicotinate.

The compositions according to the invention can advantageously contain substances for protecting the skin from the harmful effects of the sun, such as solar filters, individually or in combination, especially UV A filters and UV B filters, particularly titanium oxides and zinc oxides, oxybenzone, Parsol MCX, Parsol 1789 and filters of vegetable origin, substances for limiting the damage caused to the DNA, particularly those for limiting the fornation of thymine dimmers, such as ascorbic acid and derivatives thereof and/or Photonyl.RTM., and substances for contributing to the elimination of liver spots, such as inhibitors of melamin or tyrosinase synthesis.

The invention also relates to the method further comprising: mixing into the platelet composition substantially simultaneously with its topical application to the skin, with one or more of the ingredients selected from thrombin, epinephrine, collagen, calcium salts, and pH adjusting agents. Also useful are materials to promote degranulation or preserve platelets, additional growth factors or growth factor inhibitors, small molecule pharmaceuticals such as NSAIDS, steroids, and anti-infective agents.

In yet another aspect, the invention relates to a dermatological composition comprising: platelet releasate wherein the composition is at a pH greater than or equal to physiological pH, and wherein the composition comprises substantially no unactivated platelets.

Platelets are cytoplasmic portions of marrow megakaryocytes. They have no nucleus for replication; the expected lifetime of a platelet is some five to nine days. Platelets are involved in the hemostatic process and release several initiators of the coagulation cascade. Platelets also release cytokines involved with initiating wound healing. The cytokines are stored in alpha granules in platelets. In response to platelet to platelet aggregation or platelet to connective tissue contact, as would be expected in injury or surgery, the cell membrane of the platelet is “activated” to secrete the contents of the alpha granules. The alpha granules release cytokines via active secretion through the platelet cell membrane as histones and carbohydrate side chains are added to the protein backbone to form the complete cytolkine. Platelet disruption or fragmentation, therefore, does not result in release of the complete cytokine.

A wide variety of cytokines are released by activated platelets. Platelet derived growth factor (PDGF), transforming growth factor-beta (TGF-b), platelet-derived angiogenesis factor (PDAF) and platelet derived endothelial cell growth factor (PD-ECGF) and insulin-like growth factor (IGF) are among the cytokines released by degranulating platelets. These cytolines serve a number of different functions in the healing process, including helping to stimulate cell division at an injury site. They also work as powerful chemotactic factors for mesenchymal cells, monocytes and fibroblasts, among others.

Historically, PRP has been used to form a fibrin tissue adhesive through activation of the PRP using thrombin and calcium, as disclosed in U.S. Pat. No. 5,165,938 to Knighton, and U.S. Pat. No. 5,599,558 to Gordinier et al., incorporated in their entirety by reference herein. Activation results in release of the various cytokines and also creates a clotting reaction within various constituents of the plasma fraction. The clotting reaction rapidly forms a platelet gel (PG) which can be applied to various wound surfaces for purposes of hemostasis, sealing, and adhesion.

In another embodiment, the inventive platelet composition may comprise releasate from platelets, in addition to platelets themselves. The releasate comprises the various cytokines released by degranulating platelets upon activation. Many activators of platelets exist; these include calcium ions, thrombin, collagen, epinephrine, and adenosine diphosphate. Releasates according to the invention may be prepared according to conventional methods, including those methods described in U.S. Pat. No. 5,165,938 to Knighton, and U.S. Pat. No. 5,599,558 to Gordinier et al. The releasates alone or in a dermatologically acceptable carrier may be topically applied and/or injected into the skin.

One disadvantage of conventional releasate strategies associated with the use of PRP as PG is the use of thrombin as a preferred activator. In particular, much thrombin used in PG is bovine thrombin, which can create problems due to contamination issues regarding prions which cause Creutzfeldt-Jakob disease. Many bovine materials are suspect due to possible prion contamination, and so use of bovine thrombin is disfavored. Human pooled thrombin is likewise disfavored due to the potential of contamination with various infectious agents such as viruses, prions, bacteria and the like. Recombinant human thrombin might also be used, but may be expensive. Any of the platelets, fibroblast cells, thromin, or formulations of the invention or components thereof may be tested for the presence of prions using assays kmown in the art such as disclosed in U.S. Pat. No. 6,620,629 issued Sep. 16, 2003 and; U.S. Pat. Nos. 6,221,614; 6,617,119 issued Sep. 9, 2003; and U.S. Pat. No. 5,891,641.

It is a particular advantage of the present invention that exogenous or extra activators need not be administered to a patient. Collagen, a major component of connective tissues, is a strong activator of platelets. Thus, when the inventive platelet composition is administered to skin, platelets in the platelet composition may bind to the collagen and then be activated. This reduces or eliminates the need for administering an exogenous activator such as thrombin. The disadvantages of thrombin use have been noted above. Other strong activators, such as calcium ions, can cause severe pain, unintentional clotting, and other undesirable side effects. Thus, in an embodiment of the invention, no or substantially no exogenous activator is present or added as part of the inventive platelet composition, or is used in the preparation of the inventive platelet composition. Of course, exogenous activators may still be employed if a physician determines that they are medically necessary or desirable. Thus, the composition of the invention may consist only of platelets as the active ingredient.

The platelet composition may be prepared using any conventional method of isolating platelets from whole blood or platelet-containing blood fractions. These include centrifugal methods, filtration, affinity columns, and the like. If the platelet composition comprises PRP, then conventional methods of obtaining PRP, such as those disclosed in U.S. Pat. Nos. 5,585,007 and 5,788,662 both to Antanavich et al., incorporated herein by reference in their entirety, may be utilized.

Adjusting the pH of platelet compositions has been used to prolong the storage time of unactivated platelets, as disclosed in U.S. Pat. No. 5,147,776 to Koerner, Jr. and U.S. Pat. No. 5,474,891 to Murphy, incorporated by reference herein. pH may be adjusted using a variety of pH adjusting agents, which are preferably physiologically tolerated buffers, but may also include other agents that modify PRP pH including agents that modify lactic acid production by stored platelets. Especially useful are those pH adjusting agents that result in the pH of the platelet composition becoming greater than or equal to physiological pH. In an embodiment, the pH adjustment agent comprises sodium bicarbonate. Physiological pH, for the purposes of this invention, may be defined as being a pH ranging from about 7.35 to about 7.45. pH adjusting agents useful in the practice of this invention include bicarbonate buffers (such as sodium bicarbonate), calcium gluconate, choline chloride, dextrose (d-glucose), ethylenebis(oxyethylenenitrilo)tetraacetic acid (EGTA), 4-(2-hydroxyethyl)- 1 -piperazineethanesulfonic acid (HEPES), maleic acid, 4-morpholinepropanesulfonic acid (MOPS), 1,4-piperazinebis(ethanesulfonic acid) (PIPES), sucrose, N-tris(hydroxymethyl)methyl-2-aminoethanesulfonic acid (TES), tris(hydroxymethyl)aminomethane (TRIS BASE), tris(hydroxymethyl)aminomethane hydrochloride (TRIS.HCl), and urea. In a preferable embodiment, the pH adjusting agent is a bicarbonate buffer, more preferably, sodium bicarbonate.

### Platelet Alloimmunization

Platelets present a variety of antigens, including HLA and platelet-specific antigens. Patients transfused with platelets which are not their own often develop HLA antibodies. The patient may become refractory to all but HLA-matched platelets. When platelets are transfused to a patient with an antibody specific for an expressed antigen, the survival time of the transfused platelets may be markedly shortened. Nonimmune events may also contribute to reduced platelet survival. It is possible to distinguish immune from nonimmune platelet refractoriness by assessing platelet recovery soon after infusion, i.e., 10 - 60 minutes postinfusion platelet increment. In immune refractory states secondary to serologic incompatibility, there is poor recovery in the early postinfusion interval. In nonirmune mechanisms, i.e., splenomegaly, sepsis, fever, intravascular devices, and DIC, platelet recovery within 1 hour of infusion may be adequate while long-term survival (i.e., 24-hour survival) is reduced. Serologic tests may be helpful in selecting platelets with acceptable survival. In accordance with the present invention the platelets are preferably taken from the same patient they will be used to treat. In a similar manner the platelet releasate or any portion thereof is taken from the same patient treated with the formulation. Alternatively, the patient is treated with platelets, platelet releasate and portions thereof extracted from a donor patient tested for and found to have a close serologic match with the patient being treated.

### Cell Cultures

The cell cultures of the present invention involved the use of PRP and, for example may use PRP from the same patient the cells (e.g. fibroblast cells) being cultured were obtained from.

Example 5 below shows the cell culture with PRP therein and Example 6 shows the cell culture with three different concentrations of platelet releasate therein. The platelets may be treated in any manner to open the platelets or allow the releasate to escape. The treatment may be with an energy wave (e.g. ultra sound), agitation, temperature (heating/cooling-freezing/thawing), and chemical treatments or any combination thereof.

Many kinds of cells can be grown in culture, provided that suitable nutrients and other conditions for growth are supplied. Thus, since 1907 when Harrison noticed that nerve tissue explanted from frog embryos into dishes under clotted frog lymph developed axonal processes, scientists have made copious use of cultured tissues and cells from a variety of sources. Such cultures have been used to study genetic, physiological, and other phenomena, as well as to manufacture certain macromolecules using various fermentation techniques known in the art.

In studies of mammalian cell biology, cell cultures derived from lymph nodes, muscle, connective tissue, kidney, dermis and other tissue sources have been used. Generally speaking, the tissue sources that have been most susceptible to the preparation of cell cultures for studies are derivatives of the ancestor mesodermal cells of early development. Tissues that are the progeny of the ancestor endodermal and ectoderrnal cells have only in recent years become amenable to cell culture, of a limited sort only. The cell types derived from the endoderm and ectodern of early development include epidermis, hair, nails, brain, nervous system, inner lining of the digestive tract, various glands, and others. Essentially, long-term cultures of normal differentiated human cells, particular certain types of cells, are difficult to obtain. For various types of cartilage cultures see U.S. Pat. No. 5,902,741 issued May 11, 1999.

The cell-types subjected to a procedure of the present invention are derived from various tissues, can be of human origin or that of any other mammal, and may be of any suitable source, such as fibroblast cells, stem cells, cell from a whole pancreas, parotid gland, thyroid gland, parathyroid gland, prostate gland, lachrymal gland, cartilage, kidney, inner ear, liver, parathyroid gland, oral mucosa, sweat gland, hair follicle, adrenal cortex, urethra, and bladder, or portions or multiples thereof.

The tissue is prepared using any suitable method, such as by gently teasing apart the excised tissue or by digestion of excised tissue with collagenase via, for example, perfusion through a duct or simple incubation of, for example, teased tissue in a collagenase-containing buffer of suitable pH and tonic strength. The prepared tissue then is concentrated using suitable methods and materials, such as centrifugation through ficol gradients for concentration (and partial purification). The concentrated tissue then is resuspended into any suitable vessel, such as tissue culture glassware or plasticware. The resuspended material may include whole substructures of the tissue, cells and clusters of cells. For example, such substructures may include fibroblast cells.

The initial culture of resuspended tissue cells is a primary culture. In the initial culturing of the primary culture, the cells attach and spread on the surface of a suitable culture vessel with concomitant cell division. Subsequent to the initial culture, and usually after the realization of a monolayer of cells in the culture vessel, serially propagated secondary and subsequent cultures are prepared by dissociating the cells of the primary culture and diluting the initial culture or its succeeding cultures into fresh culture vessels, a procedure known in the art as passaging. Such passaging results in an expanded culture of cells of the originating tissue. The cell culture is passaged at suitable intervals, such as about once a week or after about two to about three cell divisions of the cultured cells. Longer intervals of two to three weeks or shorter intervals of two to three days would suffice also. For passaging the cell cultures, a dilution of the cultured cells at a ratio of from about 1:2 to about 1:100 is used.

Preferably, a ratio of from about 1:4 to about 1:50 is used. More preferably, a ratio of from about 1:4 to about 1:6 is used.

The concentrated prepared tissue, which may be in the form of free cells and/or clumps (where the clumps may constitute ordered substructures of the tissue) is resuspended at any suitable initial cell or presumptive cell density. Suitable cell densities range from about 100 cells to about 1000 cells per square centimeter of surface area of the culture vessel. For useful vessels see U.S. Pat. No. 5,274,084 issued Dec. 21, 1993 and patents and publications cited therein.

Basal media that may be used include those commercially available from Sigma Chemical Co., Life Technologies, Inc., or BioWhittaker Co. Any basal medium may be used provided that at least magnesium ion, calcium ion, zinc ion, bicarbonate ion, potassium ion, and sugar levels can be manipulated to a lower or higher concentration in the resultant medium; in particular, the magnesium ion, calcium ion, bicarbonate ion, and D-glucose levels are required at a lower concentration, zinc ion is required at the same or higher concentration, and potassium ion is required at the same or lower concentration than is usual in standard basal media.

Preferred levels of magnesium ion, as contributed by suitable magnesium salts, such as MgSO₄.multidot.7H₂O and MgCl₂.multidot.6H₂O , are between 60 and 240 mg/L; more preferred levels of magnesium salts are between 100 and 150 mg/L. Preferred levels of calcium ion, as contributed by suitable calcium salts, such as CaCl₂ .multidot.2H₂O, are between 25 and 200 mg/L; more preferred levels of calcium ion are between 40 and 125 mg/L. Preferred levels of zinc ion, as contributed by suitable zinc salts, such as ZnSO₄ .multidot.7H₂O, are between 0.1 and 0.5 mg/L; more preferred levels of zinc ion are between 0.12 and 0.40 mg/L; yet more preferred levels of zinc ion are between 0.15 and 0.20 mg/L. Preferred levels of ascorbic acid are between 30 and 125 mg/L; more preferred levels of ascorbic acid are between 40 and 100 mg/L. Preferred levels of bicarbonate ion, as contributed by suitable bicarbonate salts, such as sodium bicarbonate, are between 175 and 700 mg/L; more preferred levels of bicarbonate ion are between 300 and 400 mg/L. Preferred levels of potassium ion, as contributed by suitable potassium salts, such as potassium chloride, are between 100 and 400 mg/L; preferred levels of potassium ion are between 200 and 325 mg/L; most preferred levels of potassium ion are between 210 and 250 mg/L. Preferred levels of sugar, as contributed by a suitable sugar, such as D-glucose, are between 400 and 1800 mg/L; more preferred levels of sugar are between 600 and 1200 mg/L; most preferred levels of sugar are between 800 and 1000 mg/L. Preferred levels of human placental lactogen are between 3 and 15 .mu.g/ml; more preferred levels of human placental lactogen are between 4 and 13 .mu.g/ml; most preferred levels of human placental lactogen are between 8 and 12 .mu.g/ml. Preferred levels of insulin, as contributed by a suitable naturally-isolated, clonally-derived, or synthesized insulin, such as isolated bovine sodium-insulin, are between 50 and 20,000 ng/ml; more preferred levels of insulin are between 100 and 10,000 ng/ml; most preferred levels of insulin are between 500 and 5,000 ng/ml. (See U.S. Pat. No 6,008,047 issued Dec. 28, 1999)

The cells such as fibroblasts and keratinocytes used in accordance with the present invention may be either autogenic or allogenic. The use of allogenic cells enables the production and storage of the living skin equivalent of the present invention thereby avoiding delays in procuring grafts for the treatment of wounds. Both cell types, keratinocytes and fibroblasts could be stored frozen for months as single cell suspensions, using published methods. After thawing these cells should maintain their viability and grow readily in culture. (See U.S. Pat. No. 6,039,760 issued Mar. 21, 2000)

### Topical Formulations

The PRP obtained can be dispersed in, mixed with or combined in any fashion with a dermatologically acceptable carrier to create a topical formulation. The formulation may be an ointment, cream, lotion, oil or the like that can be placed on the skin of a human. The carrier may be comprised of natural, refined or synthetic oils. The carrier may be derived from a liquid petroleum gelled by the addition of a polyethylene resin. Composition based on aninial fats, and/or vegetable oils may be used including lard, benzoinated lard, olive oil, cottonseed oil and the like. Examples of topical formulations are described and disclosed in publications such as Remington's Pharmaceutical Sciences, (18^{th} Ed.) Mack Publishing, Co. 1990. Such formulations may comprise a preservative and bacterialcidal and/or bacterialstatic compounds as well as perfumes and coloring agents.

The topical formulations may have a buffer adder to the PRP or have the buffer in the carrier. The pH of the formulation should be balanced to obtain a pH close to physiological pH e.g. about 7.4±10% or ±5%, or 7.2 to 7.6.

The presence of other active ingredients may require a different overall pH for the formulation as some active ingredients require a particular pH range. The releasate, platelets and/or the platelet and releasate may be combined with the carrier over a wide range of concentrations, e.g. 1%, 10%, 25%, 50%, 75%, 90%, 95%, 99% carrier with the remainder being PRP, platelets, platelet releaseate or combinations thereof with or without an additional active ingredient.

### Transdermal Patch

FIG. 4 shows a first type of transdermal patch 1 which includes an impermeable support film 2 on which a matrix 3 is arranged. The active substance which is a blood cocentrate such as PRP or platelet releasate, and/or combinations thereof, is dissolved and/or dispersed in the matrix 3 which serves as a reservoir. The matrix 3, on the opposite side to the impermeable support film 2, is covered by a membrane 4 permeable to the blood concentrate, which regulates the cross-flow. This membrane may not be necessary if the degree of permeation of the blood cocentrate such as PRP or platelet releasate in the skin does not exceed the values which might cause side effects.

The degree of diffusion of the active substance (e.g. blood cocentrate such as PRP or platelet releasate) will also depend on the permeation activators, solubilizers, etc. On the free side of the permeable membrane 4 there is a layer of a contact glue 5 (adhesive layer), protected by a release strip 6. During the use of the transdermal patch the release strip 6 is pulled off and the patch is positioned on the desired part of the patient's body, exerting slight pressure. After a “start” phase the flow reaches a constant “saturation” value.

The patch 1′ of (FIG. 5), a matrix or “reservoir” 3, in which the active substance (e.g. blood concentrate such as PRP or platelet releasate) is dissolved and/or dispersed, is applied on the impermeable support film 2′. In the present case the membrane permeable to the active substance, which is used to modify the cross-flow, is missing. The matrix 3′ therefore comes into direct contact with the epidermis. The glue 5′ is located around the edge of the patch, like an adhesive ring. Everything is protected on the free side by a single release strip 6′, which is removable, as for the embodiment of FIG. 4.

The use of the “device” is as follows: the release strip 6′ is pulled off and the device is positioned on the desired part of the patient's body, exerting slight pressure.

The embodiment of FIG. 5 can be adopted in particular if the active principle interacts in an unwanted manner with the adhesive, as a result of which it is not possible to mix the adhesive 5′ and the active principles in the matrix 3′.

The pharmacological dose (e.g. blood concentrate such as PRP or platelet releasate) may be placed directly, dissolved or dispersed, into the glue, which thus also becomes a “reservoir” which may be arranged in a layer on a permeable support film. A person skilled in the art reading this disclosure will be able to modify the shape and/or structure of the patch, achieving the best result based on the therapy chosen and the site of application; or on other factors.

The differences in the structure and shape of the patch (rectangular or anatomical) may be due:

to the area of skin being treated;

to the interactions which may exist between the active principle, the glue (different types of adhesive can be used simultaneously), the support material, and other materials such as excipients, stabilizers, etc.;

to better stability on the chosen site of application;

to the dosage (the area of the patch must also increase for a higher dosage).

### Method of Manufacture of the Transdermal Patch

1. The active principle (e.g. blood concentrate such as PRP or platelet releasate) is incorporated simultaneously with the other components (stabilizer, permeation activators, etc.) in the hot adhesive solution and homogenized by stirring, until the liquid adhesive matrix or “reservoir” is obtained;

2. the liquid matrix is cooled, and acquires a “stringy” consistency;

3. the process for layering of the adhesive matrix on the support is carried out using a layering machine which is continuously connected with a drying machine, in the following phases:

the blade of a knife is mounted across the entire width of the conveyor belt of the layering machine on which the release strip is securely positioned;

the “stringy” adhesive matrix is poured in front of the blade, which, as the conveyor belt advances, distributes a uniform layer (layering) of adhesive matrix on the release strip;

the thickness of the layer is mainly determined by the distance between the edge of the knife blade and the release strip running beneath it;

the release strip, carrying the adhesive matrix, rotates inside the drying machine, in which the adhesive matrix is solidified by evaporation of the solvent, which is achieved by gradually increasing the temperature and the “ventilation”, as shown in the following Table I.

TABLE I

| | Time | | |
|---|---|---|---|
| Drying phase | (in minutes) | T° C. | Vent (rpm) |
| 1 | 15 | 40 | 700 |
| 2 | 20 | 55 | 1000 |
| 3 | 25 | 70 | 1200 |

The process described allows elimination of the solvent, preventing it from being occluded by the rapid formation of a surface crust.

When the adhesive matrix has dried, the support film (backing) is applied. This phase, called “lamination”, ends the process.

The adhesive should be inert and permeable to the active compound (e.g. blood concentrate such as PRP or platelet releasate), and the adhesive properties of which (cohesion, adhesion and interlacing) are not adversely affected by the blood concentrate such as PRP or platelet releasate itself and/or by excipients or any other material added.

### Composition of the Patch

Adhesive Matrix: Formulation

active principle: blood concentrate such as PRP or platelet releasate;

antioxidant: sodium metabisulphice, EDTA disodium salt;

solubilizing agent: a glycol;

permeation activator: fatty acids;

acrylic resin to improve the cohesive strength: cationic copolymers based on dimethylaminoethylmethacrylate and methacrylic esters;

cellulose derivatives to improve the cohesive strength: ethylcellulose;

surfactant: SDS (sodium dodecylsulphate);

pressure contact adhesive: mixture of two adhesives, A and B, in which A is a non-self-bonding acrylic contact adhesive of medium molecular weight with a high interlacing index, with a skin irritation index of 0.20, classified as “minimally irritating”, using 100% ethyl acetate as solvent; and B is a self-bonding acrylic adhesive with a high molecular weight, with moderate interlacing, with a skin irritation index of 0, classified as “non-irritating”, using a mixture of ethyl acetate, isopropanol, hexane and toluene as solvent.

### Release Strip

The release strip is a polyester film laminated with silicone on one side (that opposite of the adhesive matrix). The thickness is approximately 125 microns.

“Backing”

The “backing” is a laminated polyester film which is clear and occlusive with a themoweldable layer. The total thickness is approximately 51 microns.

Quantity of Active Principle

The quantity of blood concentrate such as PRP or platelet releasate is 5% by weight of the adhesive matrix and corresponds to 5 mg/sq cm. The major part of the blood concentrate such as PRP or platelet releasate is dispersed in the matrix. A minor part is dissolved in the matrix. The active component e.g. blood concentrate such as PRP or platelet releasate dispersed in the matrix acts as a “reservoir”, while the active component available for release and permeation is the dissolved active component.

Prophetic examples of the patch based with the active component (e.g. blood concentrate such as PRP or platelet releasate) are now given.

Two batches of patches of differentiated formulation containing active components are prepared for this purpose.

### EXAMPLE A

| | |
|---|---|
| 1) platelet releasate | 2.20% |
| 2) Solubilizing agent | 4.00% |
| 3) Acrylic resin | 29.00% |
| 4) Fatty acid 1 | 3.20% |
| 5) Fatty acid 2 | 1.60% |
| 6) Pressure-sensitive adhesive | 60.0% |

### EXAMPLE B

| | |
|---|---|
| 1) PRP | 4.99% |
| 2) EDTA | 0.025% |
| 3) Solubilizing agent | 9.96% |
| 4) Fatty acid 1 | 7.96% |
| 5) Fatty acid 2 | 3.97% |
| 6) Acrylic resin | 1.99% |
| 7) Cellulose derivative | 0.25% |
| 8) Surfactant | 20.4% |
| 9) Pressure-sensitive adhesive | 50.455% |

### Patch Devoid of Drug

There are a wide range of transdermal patches known in the art and, in general, those patches can be modified to incorporate a platelet releasate formulation of the invention. However, in an embodiment of the invention the platelet releasate is autologous to the patient being treated. To achieve such the patch can be produced without a drug (e.g. without platelet releasate) in it. The patch may have a single or multiple compartments which can be filled with the patient's own platelet releasate prior to placing the patch on the patient's skin. This can be accomplished in a number of different ways. For example, the patch may comprise a compartment which is empty or comprises a gauze, matrix or like material which readily absorbs a liquid such as releasate formulation of the invention.

The compartment may be covered by a lid which has a resealable adhesive around its edges. This makes it possible for the lid to be opened, liquid formulation is added to the container and the lid resealed. An alternative is to have a wall or portion of a wall that is comprised of a material that is self sealing when punctured with a hollow cylinder such as a hollow needle used to inject a formulation of the invention into the compartment.

In accordance with this embodiment of the invention blood is extracted from a patient and platelets of the blood concentrated. The concentrated platelets are subjected to treatment such as sonification to create a platelet releasate. The releasate is formulated to adjust the pH. The pH balanced, liquid, flowable formulation is placed in the compartment. The patient, which may be the patient from which the blood was taken, applies the patch and releasate formulation is administered transdermally. The patch may be in any shape and may, for example, be in the shape of a 3-dimensional mask shaped to fit the face of the patient or a portion of the face of the patient such as over the patient's forhead and around the patient's eyes where wrinkles are most prominent. The patch can be repeatedly applied, night after night, and worn by the patient during sleep and/or just prior to sleep. The releasate is shown to promote the growth of fibroblast cells in the cell culture of Examples 6 and 7 and fibroblast cells are essential for the young healthy appearance of skin.

An individualized transdermal patch of the invention can also be used in wound healing. The patch is prepared in a manner as indicated above and applied to a wound. The wound may also be treated with other compounds such as antibodies to aid in the treatment of infection.

### Injectable Formulations

Injectable formulations may be comprised of PRP, or platelet releasate water and buffer to balance the pH to near physiological pH e.g. about 7.4±10%, 7.4±5% or 7.2 to 7.6. Suitable formulations of the invention may be prepared using technology as taught within Remington's cited above.

Both injectable and topical formulations may further comprise fibroblast cells particularly as cultured per the present invention. Both injectable and topical formulations may further comprise PRP releasate and/or other pharmacologically active components.

### EXAMPLES

The following examples are put forth so as to provide those of ordinary skill in the art with a complete disclosure and description of how to make and use the present invention, and are not intended to limit the scope of what the inventor regard as his invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

PRP was prepared using a centrifuge unit made by Harvest (Plymouth, Mass. (Similar units are available as The Biomet GPS system, the Depuy Symphony machine and the Medtronic Magellan machine.) Approximately 55 cc of blood was drawn from the patient using a standard sterile syringe, combined with 5 cc of a citrate dextrose solution for anticoagulation, and then spun down to isolate the platelets according to the manufacturer's protocol. These platelets were then resuspended in approximately 3 cc of plasma. The resulting platelet rich plasma solution (PRP) was quite acidic and was neutralized with using approximately 0.05 cc of an 8.4% sodium bicarbonate buffer per cc of PRP under sterile conditions to approximately physiologic pH of 7.4. The PRP was not activated through addition of exogenous activators. This PRP composition is referred to herein as autologous platelet extract (APEX).

### Example 2

Fifty cc of whole blood is drawn from a patient, and then prepared according to the method of Knighton, U.S. Pat. No. 5,165,938, column 3. The PRP is activated according to Knighton using recombinant human thombin. The degranulated platelets are spun down and the releasate containing supernatant is recovered. The releasate may be optionally pH adjusted to a pH of 7.4 using sodium bicarbonate buffer.

### Example 3

Thirty ml of whole blood were drawn from a patient. A platelet composition was prepared according to Example 1 of U.S. Pat. No. 5,510,102 to Cochrum, incorporated herein by reference in its entirety, except that no alginate is added to the platelet composition.

### Example 4

Cell Cultures of Any Tissue

A researcher or clinician wishes to grow a cell culture of either fibroblasts or osteoarthritic cartilage cells. Using the technique of Example 1, an autologous platelet extract (APEX) is obtained and buffered to physiologic pH.

The cells are then isolated and grown in a media rich in the APEX in various conditions and dilutions. The APEX promotes cell differentiation and production of proteins such as collagen. The APEX may augment or promote the ability of the cells to transform into normal cells. Without intending to be limited by theory, it is hypothesized the APEX may convert the osteoarthritic cartilage cells to a more functional cell line that is reinjected into a diseased or injured joint. Alternatively, the APEX is directly introduced into an osteoarthritic joint to reverse the course of the disease. This is done under local anesthesia in a sterile manner.

### Example 5

Human Fibroblast Proliferation in Buffered Platelet Rich Plasma

Platelet rich plasma has been used to augment bone grafting and to help accelerate or initiate wound healing. Fibroblasts are important components of the wound healing process. This example shows that human fibroblast cells will proliferate more in fetal bovine serum that has been augmented with a proprietary formulation of buffered platelet rich plasma.

Human fibroblasts were isolated and then put into culture with 10% fetal bovine serum that had been augmented with a proprietary formulation of buffered platelet rich plasma (Group 1) or in 10% fetal bovine serum alone (Group 2). Initial cell counts were 25,000 in both groups.

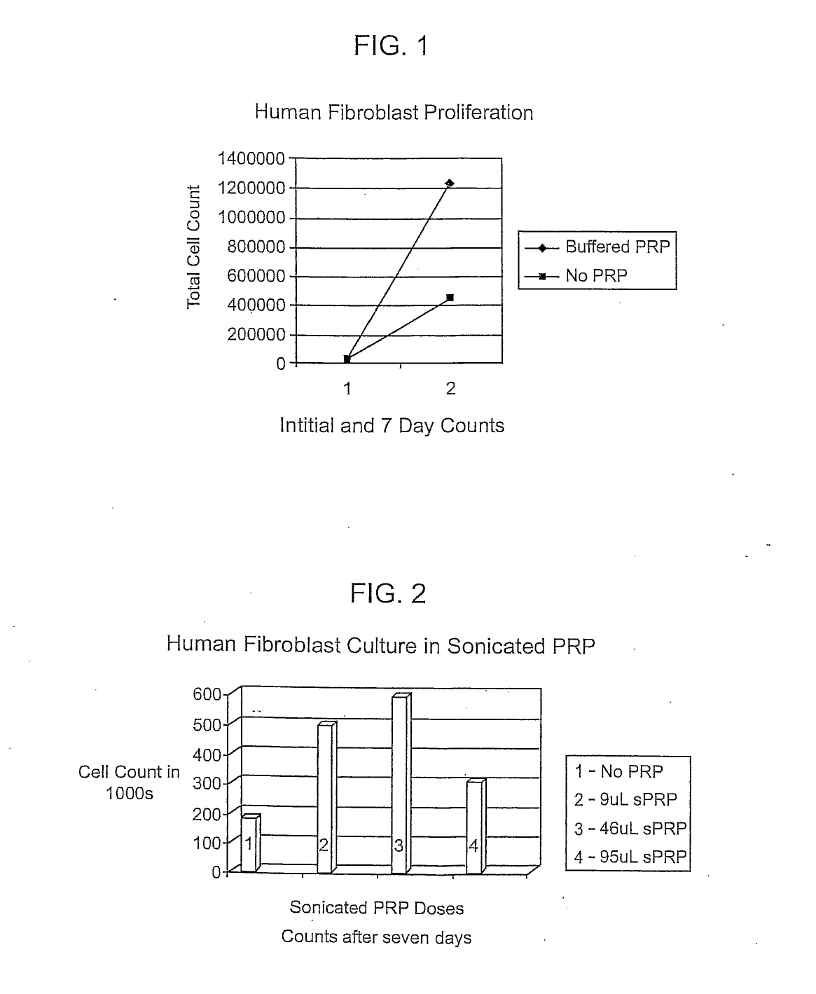

Seven days after initiating the culture experiment, the cells in each group were counted. The average total cell count in Group 1 (buffered PRP added) was 1,235,000. The average total cell count in Group 2 (No PRP) was 443,000. The group that was augmented with the buffered platelet rich plasma of the invention had 2.8 times the proliferation of the control group at seven days. (See FIG. 1)

Buffered platelet rich plasma augments human fibroblast proliferation when compared to the use of fetal bovine serum alone. This has significant implications for the use of buffered platelet rich plasma for either acute or chronic wound healing.

### Example 6

### Human Fibroblast Proliferation in Sonicated Platelet Rich Plasma

Human fibroblasts were isolated and then put into four different cultures. Three of the cultures comprised 10% fetal bovine serum that had been augmented with 9uL, 46uL, and 95uL of buffered and sonicated platelet rich plasma. The fourth served as the control and was comprised of 10% fetal bovine serum. Initial cell counts were 20,000 in both groups. Variable doses of the sonicated PRP (sPRP) were seeded with cells.

Four days after initiating the culture experiment, the cells in each of the four groups were counted and the results are shown in FIG. 2. The cell count in the control group (No PRP) was 180,000 cells. The cell counts in the sonicated PRP group were as follows: 496,000 (9 uL dose of sPRP), 592,000 (46uL dose of sPRP) and 303,000 (95uL dose of sPRP).

This experiment shows that buffered, and sonicated platelet rich plasma augments human fibroblast proliferation when compared to the use of fetal bovine serum alone.

### Example 7

### Human Fibroblast Proliferation in Sonicated Platelet Rich Plasma

Human fibroblasts were isolated and then put into twor different cultures. One of the cultures comprised 10% fetal bovine serum that had been augmented with buffered and sonicated platelet rich plasma. The other served as the control and was comprised of 10% fetal bovine serum. Initial cell counts were 20,000 in both groups.

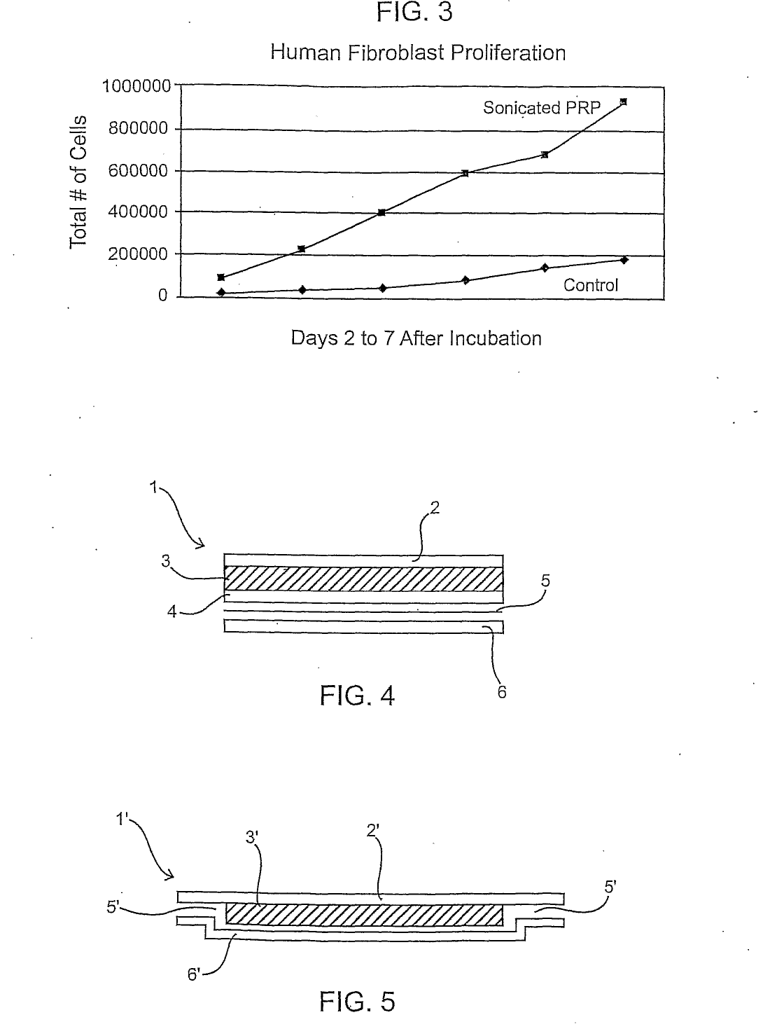

Seven days after initiating the culture experiment, the cells in each of the two groups were counted and the results are shown in FIG. 3. The cell count in the control group (No PRP) was 183,600 cells. The cell count in the sonicated PRP group was 924,800 cells.

This experiment shows that buffered, and sonicated platelet rich plasma augments human fibroblast proliferation when compared to the use of fetal bovine serum alone. These results show the ability of the platelet releasate to promote cell growth and in particular fibroblast cells which are essential to firm, young looking skin.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of treating human skin, comprising:
determining a site on skin of a human patient in need of treatment; and
topically applying to the site a composition comprising platelet-rich plasma and dermatologically acceptable carrier.

2. The method of claim 1, further comprising: rubbing the composition into the skin.

3. The method of claim 1, further comprising: allowing the composition to be absorbed into the skin wherein the composition further comprises a skin permeation enhancer.

4. The method of claim 1, wherein the site comprises facial skin.

5. The method of claim 4, wherein the platelet-rich plasma comprises platelets obtained from the patient being treated.

6. The method of claim 1, further comprising: repeating the topical application of the composition to the site about every 6 to 48 hours.

7. (canceled)

8. A method, comprising:
extracting blood from a patient;
concentrating platelets from the blood;
combining the platelets with a skin permeation enhancer and a pharmaceutically acceptable carrier for topical application to skin, thereby forming a composition.

9. The method of claim 8, wherein the patient to which the composition is applied is the same patient from which the blood is extracted.

10. The method claim 8, wherein the carrier is an injectable carrier and is combined with a component chosen from collagen, cultured cells, and mixtures thereof.

11-13. (canceled)

14. A method of culturing cells, comprising:
isolating cells from a patient;
placing the cells on a medium comprising platelet-rich plasma; and
allowing the cells to proliferate.

15. The method of claim 14, wherein the isolated cells are fibroblast cells.

16. The method of claim 14, wherein the cells are isolated from the same patient as the platelets of the platelet-rich plasma are obtained.

17. The method of claim 14, wherein the isolate cells are stem cells.

18-42. (canceled)

43. A formulation, comprising:
cells cultured in a medium comprising platelet releasate obtained from the same patient as the cells; and
a pharmaceutically acceptable carrier.

44. The formulation of claim 43, wherein the cells are human fibroblast cells and the carrier is an injectable carrier.

45. (canceled)
